(19) United States
(12) Patent Application Publication
(10) Pub. No.: US 2002/0007524 A1
(43) Pub. Date: Jan. 24, 2002

(54) Method for dyeing dry hair

(75) Inventor: Niels Henrik Sorensen, Skaevinge (DK)

Correspondence Address: NOVOZYMES NORTH AMERICA, INC., C/O NOVO NORDISK OF NORTH AMERICA, INC., 405 LEXINGTON AVENUE, SUITE 6400, NEW YORK, NY, 10174, US

(73) Assignee: Novozymes A/S, Bagsvaerd (DK)

(21) Appl. No.: 09/819,236

(22) Filed: Mar. 28, 2001

Related U.S. Application Data
(63) Continuation of application No. PCT/DK01/00166, filed on Mar. 13, 2001.
(60) Provisional application No. 60/192,688, filed on Mar. 28, 2000.

(30) Foreign Application Priority Data
Mar. 17, 2000 (DK) PA 2000 00439

(51) Int. Cl. A61K 7/13

(52) U.S. Cl. 8/405; 8/406; 8/421

(57) ABSTRACT

The present invention relates to methods for dyeing keratinous fibers, without significantly damaging the hair. According to the method of the present invention the fibers are treated in a dry state by contacting said fibers with at least one oxidoreductase and at least one dye precursor. In this way it is possible to dye, e.g. human hair, in a simple and efficient manner.

### CROSS-REFERENCE TO RELATED APPLICATIONS

This application is a continuation of PCT/DK01/00166 filed Mar. 13, 2001 and claims priority under 35 U.S.C. 119 of Danish application no. PA 2000 00439 filed Mar. 17, 2000 and U.S. application Ser. No. 60/192,688 filed Mar. 28, 2000, the contents of which are fully incorporated herein by reference.

### FIELD OF THE INVENTION

The present invention relates to a method for dyeing dry hair, more particularly, to a method for dyeing such hair by means of at least one oxidoreductase and at least one dye precursor.

### BACKGROUND OF THE INVENTION

In general hair dyeing compositions on the market today can be divided into three main groups:

temporary hair dyes,

semi-permanent hair dyes, and

permanent oxidative hair dyes.

The temporary hair dyes are only intended to change the natural hair colour for a short period of time and usually functions by depositing dyes on the surface of the hair. Such hair dyes are easy to remove with normal shampooing.

When using semi-permanent hair dyes the colour of the dyed hair can survive for five or more shampooings. This is achieved by using dyes having a high affinity for hair keratin and which is able penetrate into the interior of the hair shaft.

Permanent hair dyes are durable to sunlight, shampooing, and other hair treatments and are ordinarily refreshed periodically (about once a month) as new hair grows out. With these dyeing systems, the dyes are created directly in and on the hair. Small aromatic colourless dye precursors (e.g., p-phenylene-diamine and o-aminophenol) penetrate deep into the hair where the precursors are oxidized by an oxidizing agent into colored polymeric compounds. These colored compounds are larger than the dye precursors and are not easily washed out of the hair.

Traditionally, H₂O₂ is used in concentrations of about 1-10%, normally from about 3-6%, as the oxidizing agent. The use of H₂O₂ in dye compositions has some disadvantages as H₂O₂ damages the hair. Further, conditions frequently used for oxidative dyeing require treatment at high pH (normally around pH 9-10), which also causes damage to the hair.

To overcome the disadvantages of using H₂O₂, it has been suggested to use oxidation enzymes to replace H₂O₂.

U.S. Pat. No. 3,251,742 (Revlon) describes a method for dyeing human hair by dye formation in situ (i.e., on the hair). An oxidative enzyme is used for the colour formation reactions at a substantially neutral pH (pH 7-8.5). Laccases, tyrosinases, polyphenolases and catacolases are mentioned as suitable oxidation enzymes.

EP patent No. 504.005 (Perma S. A.) concerns compositions for hair dyeing which do not require the presence of H₂O₂ (hydrogen peroxide). The compositions comprise an enzyme capable of catalysing the formation of the polymeric dyes and also dye precursors, such as bases and couplers, in a buffer solution wherein the pH of the composition is between 6.5 and 8 and the enzyme has an optimal activity in the same pH range.

A method for enzyme-mediated dyeing of keratinous fibers, such as hair, has been described in WO 97/19999 (Novo Nordisk) and WO 97/19998 (Novo Nordisk).

The dyeing of e.g. hair is usually performed by applying the dye composition to the hair in a wetted state. Typically, the hair is washed before the dyeing process or wetted by water spray or mist.

DE 38 29 870 A1 describes the use of dye solutions for dyeing hair. The solution is applied to dry hair to give a temporary dyeing and to wet hair to give a permanent dyeing.

U.S. Pat. No. 5874618 A describes novel compounds for use in dyeing of keratinous fibers and in particular human hair. The keratinous fibers are dyed by allowing the dyeing composition to act on the dry or wet keratin fibers.

WO 97/25017 A1 describes a powdered or granulated hair dye which can be used on wet hair and on dry hair.

When hair is in a wetted state the hair fibers are swelled as compared to hair in a dry state. In this way allowing dye precursors to more easily penetrate into the hair resulting in an improved dye uptake.

The present inventor has now found that at least the same dyeing effect as when dyeing wet hair enzymatically can be obtained by applying an enzymatic dyeing composition to dry hair.

This is a commercially viable method that allows permanent dyeing of hair, with sufficient depth and permanence of color on hair without significantly damaging the hair and without the need for the consumer to wet the hair before dyeing.

### SUMMARY OF THE INVENTION

The object of the present invention is to provide an improved method for permanent dyeing of keratinous fibers e.g. human hair such that the dyeing is suitably permanent, and sufficiently mild such that it does not cause significant damage to hair.

In the context of the present invention an “improved” method for dyeing keratinous fibers means a method capable of dyeing the keratinous fibers in question faster or by the use of a smaller amount of oxidation enzyme to obtain an optimal dyeing effect, determined as ΔE*, in comparison to corresponding prior art methods of dyeing.

Further, it is also possible to use a less amount of dye precursor. This is advantageous as certain dye precursors are very unhealthy and very carcinogenic.

Further, it is desirable to be able to use a less amount of enzyme in the dyeing composition. This might make the dyeing process more economical. Further, the risk for creating airborne protein aerosols is reduced.

Even further, it is desirable to be able to dye keratinous fibers without wetting the fibers beforehand.

The present invention provides a method for dyeing keratinous fibers comprising contacting the fibers in a dry state with a dyeing composition comprising at least one oxidoreductase and at least one dye precursor for a sufficient period of time and under conditions sufficient to permit dyeing of keratinous fibers.

It is also an object of the invention to provide a use of at least one oxidoreductase for dyeing keratinous fibers in a dry state.

### BRIEF DESCRIPTION OF THE DRAWINGS

FIG. 1 shows the results of dyeing dry and wet Bertello hair.

### DETAILED DESCRIPTION OF THE INVENTION

As used in this specification and the appended claims, the singular forms “a”, “an”, and “the” include plural references unless the context clearly dictates otherwise. Thus, for example, reference to an “oxidoreductase” include mixtures of oxidoreductases. Unless defined otherwise, all technical and scientific terms used herein have the same meaning as commonly understood by one of ordinary skill in the art to which the invention applies.

The term “ingredients used in dyeing compositions” means ingredients known by the skilled person with skill in the field of formulating hair care composition to be incorporated in prior art compositions.

The present invention provides a method for dyeing keratinous fibers comprising contacting the fibers in a dry state with a dyeing composition comprising at least one oxidoreductase and at least one dye precursor for a sufficient period of time and under conditions sufficient to permit dyeing of keratinous fibers.

The term “in a dry state” means that the hair in no way has been wetted by or soaked in water prior to dyeing. The dyeing procedure may be carried out at room temperature, preferably around the optimal temperature of the enzyme, typically with from 10 to 60° C.; at a pH in the range from 3 to 10, preferably 5 to 9, especially 6 to 8; for a period of time between 10 and 60 minutes, preferably 15 to 50 minutes, especially 20 to 40 minutes.

When specific enzymes are applied example of chemical oxidizing agents are hydrogen peroxide, bromate, and other oxidants that generate hydrogen peroxide in situ such as percarbonates and perborates.

In a preferred embodiment, the concentration of said chemical oxidant such as hydrogen peroxide is sufficient to enhance depth and permanence of color on hair, relative to systems containing only oxidoreductases, but insufficient for permanent hair dyeing in the absence of oxidoreductase, and insufficient to cause significant damage to hair. According to the invention the chemical oxidizing agent is used in an amount equivalent to 0.001-1%, preferably 0.01-0.5%, calculated by weight of the dyeing formulation.

Oxidoreductases

Oxidoreductases (i.e., enzymes classified under the Enzyme Classification number E.C. 1 (Oxidoreductases) in accordance with the Recommendations (1992) of the International Union of Biochemistry and Molecular Biology (IUBMB)) are enzymes that catalyze redox reactions.

According to the invention, three types of oxidoreductases are especially contemplated:

a) Laccases or related enzymes cover enzymes which act on molecular oxygen (O₂) and yield water (H₂O) without any need for peroxide (e.g. H₂O₂),

b) Oxidases cover enzymes which act on molecular oxygen (O₂) and yield peroxide (e.g. H₂O₂), and

c) Peroxidases cover enzymes which act on peroxide (e.g. H₂O₂) and yield water (H₂O).

Preferred oxidoreductases are of microbial origin, especially recombinant and/or substantially purified enzymes without any substantial side activity. Microbial enzymes are preferred to plant and fruit enzymes as they can be produced more easily in large amounts by recombinant techniques known in the art.

The term “microbial enzyme” in the context of the present invention refers to enzymes derived from bacteria, filamentous fungi or yeasts.

Also, enzyme systems which comprise a combination of more than one enzyme among the three types of enzymes are contemplated according to the invention. The enzyme systems may e.g. consist of a laccase or a related enzyme and an oxidase; a laccase or a related enzyme and a peroxidase; a laccase or a related enzyme, an oxidase and a peroxidase; or an oxidase and a peroxidase.

Laccases and Related Enzymes

Laccases (benzenediol:oxygen oxidoreductases) (E.C. class 1.10.3.2 according to Enzyme Nomenclature (1992) Academic Press, Inc) are multi-copper containing enzymes that catalyse the oxidation of phenols. Laccase-mediated oxidations result in the production of aryloxy-radical intermediates from suitable phenolic substrates; the ultimate coupling of the intermediates so produced provides a combination of dimeric, oligomeric, and polymeric reaction products. Certain reaction products can be used to form dyes suitable for dyeing keratinous fibers (see below).

Moreover, the intermediate aryloxy-radical intermediates may themselves possess oxidative properties which may be utilised.

Suitable laccases may, for example, be derived from a strain of Polyporus sp., in particular a strain of Polyporus pinsitus (also called Trametes villosa) or Polyporus versi color, or a strain of Myceliophthora sp., e.g. M. thermophila or a strain of Rhizoctonia sp., in particular a strain of Rhizoctonia praticola or Rhizoctonia solani, or a strain of Scytalidium sp., in particular S. thermophilium, or a strain of Pyricularia sp., in particular Pyricularia oryzae, or a strain of Coprinus sp., such as a C. cinereus.

The laccase may also be derived from a fungus such as Collybia, Fomes, Lentinus, Pleurotus, Aspergillus, Neurospora, Podospora, Phlebia, e.g. P. radiata (WO 92/01046), Coriolus sp., e.g. C. hirsitus (JP 2-238885), or Botrytis.

In a preferred embodiment of the invention the laccase is derived from a strain of Myceliophthora sp., especially the Myceliophthora thermophila laccase described in WO 95/33836 (Novo Nordisk).

When using a laccase, such as the M. thermophila laccase, for keratinous fiber dyeing, the invention may be carried out at room temperature, preferably around the optimum temperature of the enzyme from 10 to 60° C., at a pH in the range from 3 to 10, preferably in the range from 5 to 9, especially in the range from 6 to 8.

Bilirubin oxidase may be derived from a strain of Myrothecium sp., such as a strain of M. verrucaria.

Peroxidases

Peroxidases are used in combination with either H₂O₂ or an oxidase to obtain the desired result Suitable peroxidases can be found within the group of enzymes acting on peroxide as acceptor, e.g. E.C. 1.11.1, especially peroxidase (E.C. 1.11.1.7).

Specific examples of suitable enzymes acting on peroxide as acceptor include peroxidases derived from a strain of the fungus Coprinus, in particular a strain of Coprinus cinereus or Coprinus macrorhizus, or derived from a strain of the bacteria Bacillus, in particular a strain of Bacillus pumilus.

Oxidases

Oxidases yielding peroxide (H₂O₂).

Suitable oxidases include glucose oxidase (E.C. 1.1.3.4), hexose oxidase (E.C. 1.1.3.5), L-amino-acid oxidase (E.C. 1.4.3.2), xylitol oxidase, galactose oxidase (E.C. 1.1.3.9), pyranose oxidase (E.C. 1.1.3.10) and alcohol oxidase (E.C. 1.1.3.13).

If an L-amino acid oxidase is used, it may be derived from a Trichoderma sp. such as Trichoderma harzianum, such as the L-amino acid oxidase described in WO 94/25574 (from Novo Nordisk A/S), or Trichoderma viride.

A suitable glucose oxidase may originate from Aspergillus Sp., such as a strain of Aspergillus niger, or from a strain of Cladosporium sp. in particular Cladosporium oxysporum.

Hexose oxidases from the red sea-weed Chondrus crispus (commonly known as Irish moss)(Sullivan and Ikawa, (1973), iochim. Biophys. Acts, 309, p. 11-22; Ikawa, (1982), Meth. in Enzymol. 89, carbohydrate metabolism part D, 145-149) oxidise a road spectrum of carbohydrates, such as D-glucose, D-galactose, altose, cellobiose, lactose, D-glucose 6-phosphate, D-mannose, 2-deoxy-D-glucose, 2-deoxy-D-galactose, D-fructose, D-glucuronic acid, and D-xylose.

Also the red sea-weed Iridophycus flaccidum produces easily extractable hexose oxidases which oxidise several different mono- and disaccharides (Bean and Hassid, (1956), J. Biol. Chem, 218, p. 425; Rand et al. (1972), J. of Food Science 37, p. 698-710).

Another suitable enzyme group is xylitol oxidase (see e.g. JP 80892242) which oxidises xylitol, D-sorbitol, D-galactitol, D-mannitol and D-arabinitol in the presence of oxygen. A xylitol oxidase can be obtained from strains of Streptomyces sp. (e.g. Streptomyces IKD472, FERM P-14339). Said enzyme has a pH optimum at 7.5 and is stable at pH 5.5 to 10.5 and at temperatures up to 65° C.

Mediators

In the present context, the term “mediator” is intended to mean an agent capable of acting as a substrate of oxidoreductases, and includes compounds commonly referred to as “enhancing agents”. Therefore, this term includes (i) compounds generally used with oxidoreductases to enhance the oxidation effect on dye precursors; (ii) compounds capable of modifying colors precursors, although incapable of providing substantial color on their own.

Examples of mediators capable of enhancing the activity of oxidoreductases include the compounds described in WO 95/01426, which is hereby incorporated by reference, and represented by the general formula I:

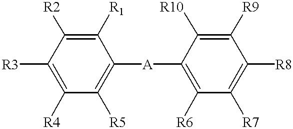

Specifically contemplated compounds within the above formula I include the following: 2,2′-azino-bis(3-ethylbenzothiazoline-6-sulfonate (ABTS); 6-hydroxy-2-naphtoic acid; 7-methoxy-2-naphtol; 7-amino-2-naphthalene sulfonic acid; 5-amino-2-naphthalene sulfonic acid; 1,5-diaminonaphthalene; 7-hydroxy-1,2-naphthimidazole; 10-methylphenothiazine; 10-phenothiazinepropionic acid (PPT); N-hydroxysuccinimide-10-phenothiazinepropionate; benzidine; 3,3′-dimethylbenzidine; 3,3′-dimethoxybenzidine; 3,31,5,51-tetramethylbenzidine; 4′-hydroxy-4-biphenylcarboxylic acid; 4-amino-4′-methoxystilbene; 4,4′-diaminostilbene-2,2′-disulfonic acid; 4,4′-diaminodiphenylamine; 2,7-diaminofluorene; 4,4′-dihydroxy-biphenylene; triphenylamine; 10-ethyl-4-phenothiazinecarboxylic acid; 10-ethylphenothiazine; 10-propylphenothiazine; 10-isopropylphenothiazine; methyl-10-phenothiazinepropionate; 10-phenylphenothiazine; 10-allylphenothiazine; 10-phenoxazinepropionic acid (POP); 10-(3-(4-methyl-1-piperazinyl)propyl)phenothiazine; 10-(2-pyrrolidinoethyl)phenothiazine; 10-methylphenoxazine; iminostilbene; 2-(p-aminophenyl)-6-methylbenzothiazole-7-sulfonic acid; N-benzylidene-4-biphenylamine; 5-amino-2-naphthalenesulfonic acid; 7-methoxy-2-naphtol; 4,4′-dihydroxybenzophenone; N-(4-(dimethylamino)benzylidene)-p-anisidine; 3-methyl-2-benzothiazolinone(4-(dimethylamino)benzylidene)hydrazone; 2-acethyl-10-methylphenothiazine; 10-(2-hydroxyethyl)phenothiazine; 10-(2-hydroxyethyl)phenoxazine; 10-(3-hydroxypropyl)phenothiazine; 4,4′-dimethoxy-N-methyl-diphenylamine, and vanillin azine.

Other mediators contemplated include 4-hydroxybenzoic acid, L-tyrosine, syringate acids, ferulic acid, sinapic acid, chlorogenic acid, caffeic acid and esters thereof.

Still further examples include organic compounds described in WO 96/10079, which is hereby incorporated by reference, and represented by the general formula II:

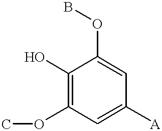

Specific compounds covered by the above formula II are acetosyringone, syringaldehyde, methylsyringate, syringic acid, ethylsyringate, propylsyringate, butylsyringate, hexylsyringate, octylsyringate and ethyl 3-(4-hydroxy-3,5-dimethoxyphenyl)acrylate.

WO 99/36034, WO 99/36035, WO 99/36036, WO 99/36037, wO 99/36038, WO 99/36039, WO 99/36040, WO 9/36041, WO 99/36042, WO 99/36043, WO 99/36044, WO 99/36045 and WO 99/36046 in the name of L'Oreal discloses different kind of oxidizing dyes (developed substances or oxidation bases or precursors) and coupling components (coupling agents) which can also be used according to the present invention and which are hereby incorporated by reference.

Precursors

Precursors are defined herein as mediators that are converted into colored compounds by oxidation. Precursors may be compounds belonging to one of three major chemical families: the diamines, aminophenols (or aminonaphtols) and the phenols.

Furthermore, a number of indole or indoline derivative precursors are disclosed in WO 94/00100, and other suitable benzoic acid precursors are disclosed in WO 98/15257 (Novo Nordisk). Said precursors mentioned in these documents are hereby incorporated herein by reference.

Examples of such suitable precursors include compounds from the group comprising p-phenylene-diamine (PPD), p-toluylenediamine, chloro-p-phenylenediamine, p-aminophenol, o-aminophenol and 3,4-diaminotoluene, 2-methyl-1,4-diaminobenzene, 4-methyl-o-phenylenediamine, 2-methoxy-p-phenylenediamine, 2-chloro-1,4-diamino-benzene, 4-amino diphenylamine, 1-amino-4-β-methoxyethylamino-benzene, 1-amino-4-bis-(β-hydroxyethyl)aminobenzene, 1-3-diamino-benzene, 2-methyl-1,3-diamino-benzene, 2,4-diaminotoluene, 2,6-diaminopyridine, 1-hydroxy-2-aminobenzene, 1-hydroxy-3-amino-benzene, 1-methyl-2-hydroxy-4-aminobenzene, 1-methyl-2-hydroxy-4-β-hydroxyethylamino-benzene, 1-hydroxy-4-amino-benzene, 1-hydroxy-4-methylamino-benzene, 1-methoxy-2,4-diamino-benzene, 1-ethoxy-2,3-diamino-benzene, 1-β-hydroxyethyloxy-2,4-diamino-benzene, phenazines, such as 4,7-phenazinedicarboxylic acid, 2,7-phenazinedicarboxylic acid, 2-phenazinecarboxylic acid, 2,7-diaminophenazine, 2,8-diaminophenazine, 2,7-diamino-3,8-dimethoxyphenazine, 2,7-diamino-3-methoxyphenazine, 2,7-diamino 3-methoxyphenazine, 3-dimethyl 2,8-phenazinediamine, 2,2′-[(8-amino-7-methyl-2-phenazinyl)imino]bis-ethanol, 2,2′-[(8-amino-7-methoxy-2-phenazinyl)imino]bis-ethanol, 2,2′-[(8-amino-7-chloro-2-phenazinyl)imino]bis-ethanol, 2-[(8-amino-7-methyl-2-phenazinyl)amino]-ethanol, 2,2′-[(8-amino-2-phenazinyl)imino]bis-ethanol, 3-amino-7-(dimethylamino)-2,8-dimethyl-5-phenylchloride, 9-(diethylamino)-benzo[a]phenazine-1,5-diol, N-[8-(diethylamino)-2-phenazinyl]-methanesulfonamide, N-(8-methoxy-2-phenazinyl)-methanesulfonamide, N,N,N′,N′-tetramethyl-2,7-phenazinediamine, 3,7-dimethyl-2-phenazinamine, p-amino benzoic acids, such as p-amino benzoic acid ethyl, p-amino benzoic acid glycerid, p-amino benzoic acid isobutyl, p-dimethylamino benzoic acid amil, p-dimethylamino benzoic acid octyl, p-diethoxy amino enzoic amil, p-dipropoxy amino benzoic acid ethyl, acetylsalicylic acid, and isatin derivatives, such as 2,3-diamino benzoic acid, and mixtures of the above precursors.

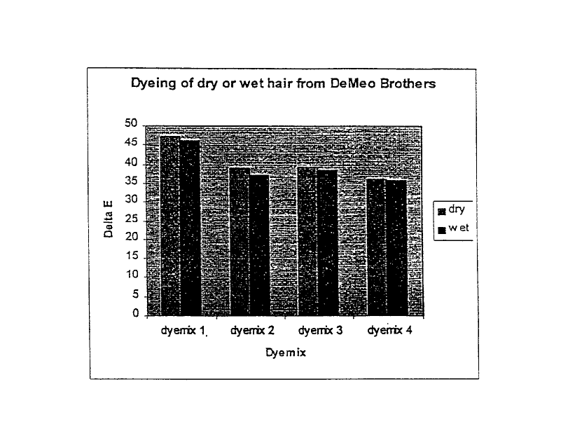

Specifically contemplated mixtures of mediators include the mixtures published in DK patent appln. no. 358/98 (see especially the table in FIGS. 1 to 3).

The following list of precursors or oxidation bases is added to the list above:

The oxidation bases can in particular be selected among para-phenylenediamines, double bases, para-aminophenols, ortho-aminophenols and heterocyclic oxidation bases.

Among the para-phenylenediamines suitable as oxidation bases in the dye compositions according to the invention, the following compounds of the formula (I) and their addition salts with an acid can in particular be mentioned:

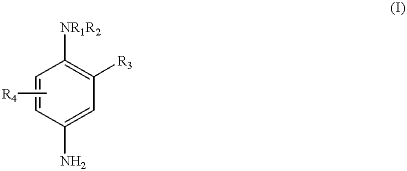

in which

R₁ is a hydrogen atom, C₁-C₄-alkyl, C₁-C₄-monohydroxyalkyl, C₂-C₄-polyhydroxyalkyl, (C₁-C₄)alkoxy(C₁-C₄)alkyl, C₁-C₄-alkyl substituted with a nitrogen-containing group, phenyl or 4′-aminophenyl;

R₂ is a hydrogen atom, C₁-C₄-alkyl, C₁-C₄monohydroxyalkyl, C₂-C₄polyhydroxyalkyl, (C₁-C₄)alkoxy(C₁-C₄)alkyl or C₁-C₄alkyl substituted with a nitrogen-containing group;

R₃ is a hydrogen atom, a halogen atom such as chlorine, bromine, iodine or fluorine, C₁-C₄alkyl, C₁-C₄monohydroxyalkyl, C₁-C₄hydroxyalkoxy, C₁-C₄acetylaminoalkoxy, Cₗ-C₄mesylaminoalkoxy or C₁-C₄carbamoylaminoalkoxy,

R₄ is a hydrogen atom, a halogen atom or C₁-C₄-alkyl.

Among the nitrogen-containing groups in the above formula (I), amino, mono(C₁-C₄)alkylamino, di(C₁-C₄)alkylamino, tri(C₁-C₄) alkylamino, monohydroxy(C₁-C₄)alkylamino, imidazolinium and ammonium can in particular be mentioned.

More particularly among the para-phenylenediamines of the above formula (I), the following para-phenylenediamines can be mentioned: para-phenylenediamine, paratoluylenediamine, 2-chloro para-phenylenediamine, 2,3-dimethyl para-phenylene-diamine, 2,6-dimethyl para-phenylenediamine, 2,6-diethyl paraphenylenediamine, 2,5-dimethyl para-phenylenediamine, N,N-dimethyl para-phenylenediamine, N,N-diethyl para-phenylenediamine, N,N-dipropyl para-phenylenediamine, 4-amino N,N-diethyl 3-methyl aniline, N,N-bis(β-hydroxy-ethyl) para-phenylenediamine, 4-N,N-bis-(β-hydroxyethyl)amino 2-methyl aniline, 4-N,N-bis-(β-hydroxyethyl)amino 2-chloro aniline, 2-3-hyroxyethyl para-phenylenediamine, 2-fluoro paraphenylenediamine, 2-isopropyl para-phenylene-diamine, N-(β-hydroxypropyl) para-phenylenediamine, 2-hydroxymethyl paraphenylenediamine, N,N-dimethyl 3-methyl para-phenylenediamine, N,N-(ethyl, β-hydroxyethyl) para-phenylenediamine, N-(β,γ-dihydroxypropyl) para-phenylenediamine, N-(4′-aminophenyl) para-phenylenediamine, N-phenyl para-phenylene-diamine, 2-β-hydroxyethyloxy para-phenylenediamine, 2-β-acetylaminoethyloxy para-phenylenediamine, N-(β-methoxyethyl) para-phenylenediamine and their addition salts with an acid.

Among the para-phenylenediamines of the above formula (I), the following are especially preferred: para-phenylenediamine, paratoluylenediamine, 2-isopropyl para-phenylenediamine, 2-1-hydroxyethyl para-phenylenediamine, 2-β-hydroxyethyloxy para-phenylenediamine, 2,6-dimethyl para-phenylenediamine, 2,6-diethyl para-phenylenediamine, 2,3-dimethyl para-phenylenediamine, N,N-bis-(β-hydroxyethyl) para-phenylenediamine, 2-chloro para-phenylenediamine, 2-β-acetylaminoethyloxy para-phenylenediamine and their addition salts with an acid.

By double bases is according to the invention meant such compositions which include at least two aromatic nuclei carrying s amino and/or hydroxyl groups.

Among the double bases suitable as oxidation bases in the dye compositions according to the invention, the compounds of the following formula (II) and their addition salts with an acid can in particular be mentioned:

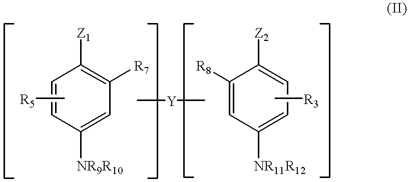

in which

Z₁ and Z₂, which are identical or differ, represent a hydroxyl gruppe or —NH₂, which can be substituted with a C₁-C₄alkyl group or with a bridging group Y;

the bridging group Y is a linear or branched alkylene chain with 1 to 14 carbon atoms, which can be interrupted or terminated by one or more nitrogen-containing groups and/or one or more hetero atoms, such as oxygen, sulphur or nitrogen atoms, and optionally be substituted with one or more hydroxyl groups or C₁-C₆-alkoxy groups;

R₅ and R₆ represents a hydrogen or halogen atom, C₁-C₄alkyl, C₁-C₄mono-hydroxyalkyl, C₂-C₄polyhydroxyalkyl, C₁-C₄aminoalkyl or a bridging group Y;

R₇, R₈, R₉, R₁₀, R₁₁ and R₁₂, which are identical or differ, represent a hydrogen atom, a bridging group Y or a C₁-C₄alkyl group;

whereby it should be understood that the compounds of the formula (II) only include a single bridging group Y per molecule.

Among nitrogen-containing groups of the above formula (II), the following can in particular be mentioned: amino, mono(C₁-C₄)alkylamino, di(C₁-C₄)alkyl-amino, tri(C₁-C₄)alkylamino, monohydroxy(C₁-C₄)alkylamino, imidazolinium and ammonium.

Among the double bases of the above formula (II), the following can more particularly be mentioned: N,N=-bis-(β-hydroxyethyl) N,N′-bis-(4′-aminophenyl) 1,3-diamino propanol, N,N=-bis-(β-hydroxyethyl) N,N=-bis-(4′-aminophenyl) ethylenediamine, N,N=-bis-(4-aminophenyl) tetramethylenediamine, N,N′-bis-(β-hydroxyethyl) N,N′-bis-(4-aminophenyl) tetramethylenediamine, N,N′-bis-(4-methylaminophenyl) tetramethylenediamine, N,N′-bis-(ethyl) N,N′-bis-(4′-amino, 3-methylphenyl) ethylenediamine, 1,8-bis-(2,5-diaminophenoxy)-3,5-dioxaoctane and their addition salts with an acid.

Particularly preferred double bases of the formula (II) are N,N′-bis-(β-hydroxyethyl) N,N′-bis-(4′-aminophenyl) 1,3-diamino propanol, 1,8-bis-(2,5-diamino-phenoxy)-3,5-dioxaoctane or one of their addition salts with an acid.

Among the para-aminophenols suitable as oxidation bases in the dye compositions according to the invention, the compounds of the following formula (III) and their addition salts with an acid can especially be mentioned:

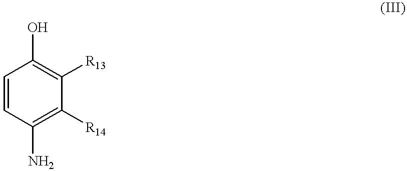

in which

R₁₃ represents a hydrogen or halogen atom, C₁-C₄alkyl, C₁-C₄monohydroxyalkyl, (C₁-C₄)alkoxy(C₁-C₄)alkyl, C₁-C₄aminoalkyl or (C₁-C₄) hydroxyalkyl (C₁-C₄) aminoalkyl, R₁₄ represents a hydrogen or halogen atom, C₁-C₄alkyl, C₁-C₄monohydroxyalkyl, C₂-C₄POlyhydroxyalkyl, C₁-C₄aminoalkyl, C₁-C₄cyanoalkyl or (C₁-C₄)alkoxy(C₁-C₄)alkyl, whereby it should be understood that at least one of the groups R₁₃ or R₁₄ represents a hydrogen atom.

Among the para-aminophenols of the above formula (III), the following can in particular be mentioned: para-aminophenol, 4-amino 3-methyl phenol, 4-amino 3-fluoro phenol, 4-amino 3-hydroxymethyl phenol, 4-amino 2-methyl phenol, 4-amino 2-hydroxymethyl phenol, 4-amino 2-methoxymethyl phenol, 4-amino 2-aminomethyl phenol, 4-amino 2-(β-hydroxyethyl aminomethyl) phenol, 4-amino 2-fluoro phenol and acid addition salts thereof.

Among the ortho-aminophenols suitable as oxidation bases in the dye compositions according to the invention, the following can in particular be mentioned: 2-amino phenol, 2-amino 5-methyl phenol, 2-amino 6-methyl phenol, 5-acetamido 2-amino phenol and acid addition salts thereof.

Among the heterocyclic bases suitable as oxidation bases in the dye compositions according to the invention, the following can in particular be mentioned: pyridine derivatives, pyrimidine derivatives, pyrazole derivatives, pyrazolo-pyrimidine derivatives and acid addition salts thereof.

Among the pyridine derivatives, the compositions described for instance in the patents GB-PS 1 026 978 and GB-PS 1 153 196 can in particular be mentioned: 2,5-diamino pyridine, 2-(4-methoxyphenyl)amino 3-amino pyridine, 2,3-diamino 6-methoxy pyridine, 2-(β-methoxyethyl)amino 3-amino 6-methoxy pyridine, 3,4-diamino pyridine and the addition salts thereof.

Among the pyrimidine derivatives, the compositions described for instance in the German patent DE 2 359 399 or the Japanese patents JP 88-169 571 and JP 91-333 495 or in the Patent Application WO 96/15765 can in particular be mentioned: 2,4,5,6-tetra-aminopyrimidine, 4-hydroxy 2,5,6-triaminopyrimidine, 2-hydroxy 4,5,6-triaminopyrimidine, 2,4-dihydroxy 5,6-diaminopyrimidine, 2,5,6-triaminopyrimidine and their addition salts with an acid.

Among the pyrazole derivatives, the compounds described for instance in the patents DE 3 843 892 and DE 4 133 957 and in the Patent Applications WO 94/08969, WO 94/08970, FR-A-2 733 749 and DE 195 43 988 can in particular be mentioned: 4,5-diamino 1-methyl pyrazole, 3,4-diamino pyrazole, 4,5-diamino 1-(4′-chlorobenzyl) pyrazole, 4,5-diamino 1,3-dimethyl pyrazole, 4,5-diamino 3-methyl 1-phenyl pyrazole, 4,5-diamino 1-methyl 3-phenyl pyrazole, 4-amino 1,3-dimethyl 5-hydrazino pyrazole, 1-benzyl 4,5-diamino 3-methyl pyrazole, 4,5-diamino 3-tert-butyl 1-methyl pyrazole, 4,5-diamino 1-tert-butyl 3-methyl pyrazole, 4,5-diamino 1-(β-hydroxyethyl) 3-methyl pyrazole, 4,5-diamino 1-ethyl 3-methyl pyrazole, 4,5-diamino 1-ethyl 3-(4′-methoxyphenyl) pyrazole, 4,5-diamino 1-ethyl 3-hydroxymethyl pyrazole, 4,5-diamino 3-hydroxymethyl 1-methyl pyrazole, 4,5-diamino 3-hydroxymethyl 1-isopropyl pyrazole, 4,5-diamino 3-methyl 1-isopropyl pyrazole, 4-amino 5-(2′-aminoethyl)amino 1,3-dimethyl pyrazole, 3,4,5-triamino pyrazole, 1-methyl 3,4,5-triamino pyrazole, 3,5-diamino 1-methyl 4-methylamino pyrazole, 3,5-diamino 4-(β-hydroxyethyl)amino-1-methyl pyrazole and their acid addition salts.

Among the pyrazolo pyrimidine derivatives, the following can in particular be mentioned: the pyrazolo-[1,5-a]-pyrimidines of the formula (IV) shown below, their addition salts with an acid or base and their tautomeric forms when a tautomeric equilibrium exists:

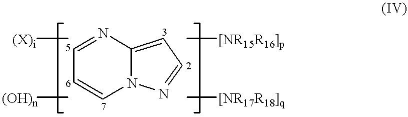

in which

R₁₅, R₁₆, R₁₇ and R₁₈, which are identical or differ, are a hydrogen atom, C₁-C₄alkyl, aryl, C₁-C₄hydroxyalkyl, C₂-C₄polyhydroxyalkyl, (C₁-C₄) alkoxy(C₁-C₄) alkyl, C₁-C₄aminoalkyl (where the amine can be protected by an acetyl, ureido or sulfonyl group), (C₁-C₄)alkylamino (C₁-C₄)alkyl, di-[(C₁-C₄)alkyl] amino C₁-C₄alkyl (where the dialkyl groups can form a carbon ring or a heterocyclic ring with 5 or 6 members), hydroxy-C₁-C₄alkyl or di-[hydroxy(C₁-C₄)alkyl] -amino C₁-C₄alkyl;

the groups X, which are identical or differ, represent a hydrogen atom, C₁-C₄alkyl, aryl, C₁-C₄hydroxyalkyl, C₂-C₄polyhydroxyalkyl, amino C₁-C₄alkyl, (C₁-C₄) alkyl (C₁-C₄) aminoalkyl, di-[(C₁-C₄)alkyl] aminoC₁-C₄alkyl (where the dialkyl groups can form a carbon ring or a heterocyclic ring with 5 or 6 members), hydroxy (C₁-C₄)alkyl or di-[hydroxy(C₁-C₄)alkyl]amino-C₁-C₄alkyl, amino, C₁-C₄alkyl or di-[(C₁-C₄)alkyl]-amino, a halogen atom, a carboxylic acid group or a sulfonic acid group;

i is 0, 1, 2 or 3;

p is 0 or 1;

q is 0 or 1;

n is 0 or 1;

with the proviso that

the sum p+q differs from 0;

when p+q is 2, n has the value 0, and the groups NR₁₅Rₗ₆ and NR₁₇R₁₈ occupy the positions (2,3); (5,6); (6,7); (3,5) or (3,7);

when p+q is 1, n has the value 1, and the group NR₁₅R₁₆ (or NR₁₇R₁₈) and the group OH occupy the positions (2,3); (5,6); (6,7); (3,5) or (3,7).

When the pyrazolo-[1,5-a]-pyrimidines of the above formula (IV) are such which include a hydroxyl group in one of the positions 2, 5 or 7 in the α-position to a nitrogen atom, a tautomeric equilibrium exists which for instance can be indicated by the following reaction scheme.

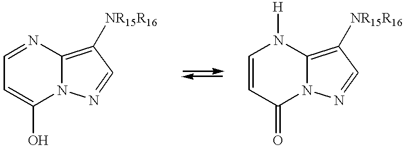

Among the pyrazolo-[1,5-a]-pyrimidines of the above formula (IV), the following can be mentioned in particular:

pyrazolo-[1,5-a]-pyrimidine-3,7-diamine;

2,5-dimethyl pyrazolo-[1,5-a]-pyrimidine-3,7-diamine;

pyrazolo-[1,5-a]-pyrimidine-3,5-diamine;

2,7-dimethyl pyrazolo-[1,5-a]-pyrimidine-3,5-diamine;

3-amino pyrazolo-[1,5-a]-pyrimidine-7-ol;

3-amino pyrazolo-[1,5-a]-pyrimidine-5-ol;

2-(3-amino pyrazolo-[1,5-a]-pyrimidine-7-ylamino)-ethanol;

2-(7-amino pyrazolo-[1,5-a]-pyrimidine-3-ylamino)-ethanol;

2-[(3-amino-pyrazolo[1,5-a]pyrimidine-7-yl)-(2-hydroxyethyl)-amino]ethanol;

2-[(7-amino-pyrazolo[1,5-a]pyrimidine-3-yl)-(2-hydroxyethyl)-amino]ethanol;

5,6-dimethyl pyrazolo-[1,5-a]-pyrimidine-3,7-diamine;

2,6-dimethyl pyrazolo-[1,5-a]-pyrimidine-3,7-diamine;

2,5, N 7, N 7-tetramethyl pyrazolo-[1,5-a]-pyrimidine-3,7-diamine;

and their addition salts and tautomeric forms, provided a tautomeric equilibrium exists.

The pyrazolo-[1,5-a]-pyrimidines of the above formula (IV) can be prepared by way of cyclisation of an aminopyrazole according to the syntheses described in the following references:

EP 628559 BEIERSDORF-LILLY

R. Vishdu, H. Navedul, Indian J. Chem., 34b (6), 514, 1995.

N. S. Ibrahim, K. U. Sadek, F. A. Abdel-Al, Arch. Pharm., 320, 240, 1987.

R. H. Springer, M. B. Scholten, D. E. O'Brien, T. Novinson, J. P. Miller, R. K. Robins, J. Med. Chem., 25, 235, 1982.

T. Novinson, R. K. Robins, T. R. Matthews, J. Med. Chem., 20, 296, 1977.

U.S. Pat. No. 3,907,799 ICN PHARMACEUTICAL

The pyrazolo-[1,5-a]-pyrimidines of the above formula (IV) can furthermore be produced by cyclisation from a hydrazine according to the syntheses described in the following references:

A. McKillop, R. J. Kobilecki, Heterocycles, 6(9), 1355, 1977.

E. Alcade, J. De Mendoza, J.M. Marcia-Marquina, C. Almera, J. Elguero, J. Heterocyclic Chem., 11(3), 423, 1974.

K. Saito, I. Hori, M. Higarashi, H. Midorikawa, Bull. Chem. Soc. Japan, 47(2), 476, 1974.

The oxidation base or bases represent preferably between approximately 0.0005 and approximately 12% by weight of the total weight of the dye composition according to the invention, especially between approximately 0.005 and approximately 6% by weight.

Modifiers

By including compounds referred to as modifiers (also known as couplers or coupling agents) in the dyeing composition, a number of color tints can be obtained. Cathecol and Resorcinol are examples of such modifiers. Modifiers are defined as a class of mediators that provides little color when oxidized in the absence of other mediators, but can significantly modify the generated colors when used in the presence of other mediators, in particular precursors.

Preferably, at least one modifier is used in combination with the oxidoreductase in the method of the invention, thereby allowing a number of color tints to be obtained. In general, modifiers are used in dyeing methods, as the colors resulting from hair dyeing without a modifier are usually unacceptable for most people.

Modifiers are typically m-diamines, m-aminophenols, or polyphenols or a combination thereof. The modifier reacts with mediators in the presence of the oxidative enzyme, converting it into a colored compound.

Examples of modifiers include m-phenylene-diamine, 2,4-diaminoanisole, 1-hydroxynaphthalene(a-naphthol), 1,4-dihydroxybenzene(hydroquinone), 1,5-dihydroxynapthalene, 1,2-dihydroxybenzene(pyrocatechol), 1,3-dihydroxybenzene (resorcinol), 1,3-dihydroxy-2-methylbenzene, 1,3-dihydroxy-4-chlorobenzene(4-chlororesorcinol), 1,2,3,trihydroxybenzene, 1,2,4-trihydroxybenzene, 1,2,4-trihydroxy-5-methylbenzene and 1,2,4-trihydroxytoluene, and mixtures thereof.

The following list of coupling agents is added to the list above:

The coupling agent or coupling agents suitable in the dye compositions according to the invention are such which are conventionally used in oxidation dye composition, viz. metaphenylene diamines, metaaminophenols, metadiphenols, heterocyclic coupling agents and their addition salts with an acid.

These coupling agents can especially be selected among 2-methyl-5-amino-phenol, 5-N-(β-hydroxyethyl)-amino-2-methyl-phenol, 3-amino-phenol, 1,3-dihydroxybenzene, 1,3-dihydroxy-2-methyl-benzene, 4-chloro-1,3-dihydroxy-benzene, 2,4-diamino-1-(β-hydroxyethyloxy)-benzene, 2-amino-4-(β-hydroxyethylamino)-1-methoxy-benzene, 1,3-diamino-benzene, 1,3-bis-(2,4-diaminophenoxy)-propane, sesamol, a-naphtol, 6-hydroxy-indole, 4-hydroxy-indole, 4-hydroxy-N-methyl-indole, 6-hydroxy-indolin, 2,6-dihydroxy-4-methyl-pyridine, 1-H-3-methyl-pyrazole-5-on, 1-phenyl-3-methyl-pyrazole-5-one, 2,6-dimethyl-pyrazolo-[1,5-b]-1,2,4-triazole, 2,6-dimethyl-[3,2-c]-1,2,4-triazole, 6-methyl-pyrazolo-[1,5-a]-benzimidazole and acid addition salts thereof.

The meta-aminophenol or meta-aminophenols applicable as coupling agents in the ready-to-use dye composition according to the invention is/are preferably selected from compounds of the following formula (III) and acid addition salts thereof:

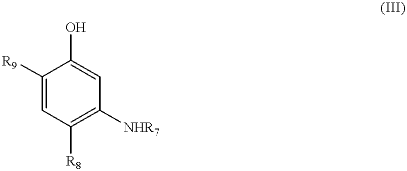

in which

R₇ is a hydrogen atom, C₁-C₄-alkyl, C₁-C₄monohydroxyalkyl or C₂-C₄polyhydroxyalkyl,

R₈ is a hydrogen atom, C₁-C₄alkyl, C₁-C₄alkoxy or a halogen atom selected from chlorine, bromine and fluorine,

R₉ represents a hydrogen atom, C₁-C₄alkyl, C₁-C₄alkoxy, C₁-C₄mono-hydroxyalkyl, C₂-C₄polyhydroxyalkyl, C₁-C₄monohydroxyalkoxy or C₂-C₄poly-hydroxyalkoxy.

Among the meta-aminophenols of the above formula (III), the following can be mentioned in particular: meta-aminophenol, 5-amino-2-methoxy phenol, 5-amino-2-(β-hydroxyethyloxy) -phenol, 5-amino-2-methyl phenol, 5-N-(β-hydroxyethyl)amino-2-methyl phenol, 5-N-(P-hydroxyethyl)amino-4-methoxy-2-methyl phenol , 5-amino-4-methoxy-2-methyl phenol, 5-amino-4-chloro-2-methyl phenol, 5-amino-2,4-dimethoxy phenol, 5-(γ-hydroxypropylamino)-2-methyl phenol and acid addition salts thereof.

The meta-phenylenediamine or meta-phenylenediamines applicable as coupling agents in the ready-to-use dye composition according to the invention is/are preferably selected from compounds of the following formula (IV) and acid addition salts thereof:

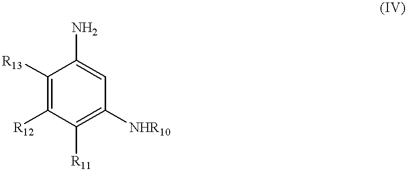

in which

R₁₀ represents a hydrogen atom, C₁-C₄alkyl, C₁-C₄monohydroxyalkyl or C₂-C₄polyhydroxyalkyl;

R₁₁ and R₁₂, which are identical or differ, each represents a hydrogen atom, C₁-C₄alkyl, C₁-C₄monohydroxyalkoxy or C₂-C₄polyhydroxyalkoxy;

R₁₃ represents a hydrogen atom, C₁-C₄alkoxy, C₁-C₄aminoalkoxy, C₁-C₄mono-hydroxyalkoxy, C₂-C₄polyhydroxyalkoxy or 2,4-diaminophenoxyalkoxy.

Among the meta-phenylenediamines of the above formula (IV) the following can in particular be mentioned: 2,4-diamino-benzene, 3,5-diamino-1-ethyl-2-methoxybenzene, 3,5-diamino-2-methoxy-1-methyl benzene, 2,4-diamino-1-ethoxybenzene, 1,3-bis-(2,4-diaminophenoxy) propane, bis-(2,4-diaminophen-oxy)-methane, 1-(β-aminoethyloxy)-2,4-diamino-benzene, 2-amino-1-(β-hydroxyethyloxy)-4-methylamino-benzene, 2,4-diamino-1-ethoxy 5-methylbenzene, 2,4-diamino-5-(β-hydroxyethyloxy)-1-methylbenzene, 2,4-diamino-1-(β,γ-dihydroxy-propyloxy) benzene, 2,4-diamino-1-(β-hydroxyethyloxy)-benzene, 2-amino-4-N-(β-hydroxyethyl)-amino-1-methoxy-benzene and acid addition salts thereof.

The meta-diphenol or meta-diphenols applicable as coupling agents in the ready-to-use dye composition according to the invention is/are preferably selected from the compounds of the following formula (V) and acid addition salts thereof:

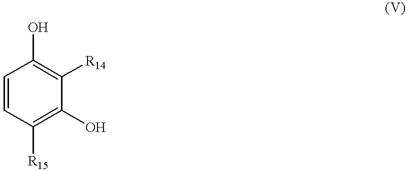

in which

R₁₄ and R15, which are identical or differ, each represents a hydrogen atom, C₁-C₄alkyl or a halogen atom selected from chlorine, bromine and fluorine.

Among the meta-diphenols of the above formula (V), the following can in particular be mentioned: 1,3-dihydroxy-benzene, 2-methyl-1,3-dihydroxy-benzene, 4-chloro-1,3-dihydroxy-benzene, 2-chloro-1,3-dihydroxybenzene, and acid addition salts thereof.

Among the heterocyclic coupling agents applicable in the ready-to-use dye composition according to the invention, derivatives of benzimidazole, derivatives of benzomorpholine, derivatives of sesamol, pyrazolo-azol derivatives, pyrrolo-azole derivatives, imidazolo-azole derivatives, pyrazolo-pyrimidine derivatives, derivatives of pyrazoline-3,5-diones, pyrrolo-[3,2-d]oxazole derivatives, pyrazolo-[3,4-d]-thiazole derivatives, thiazolo-azole S-oxide derivatives, thiazolo-azole S,S-dioxide derivatives and their addition salts with an acid can in particular be mentioned.

Among the benzimidazole derivatives applicable as heterocyclic coupling agents in the dye composition according to the invention, the compounds of the following formula (I) and their acid addition salts can in particular be mentioned:

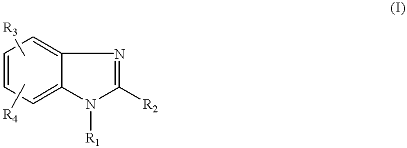

in which:

R₁ is a hydrogen atom or C₁-C₄-alkyl,

R₂ is a hydrogen atom, C₁-C₄alkyl or phenyl,

R₃ is a hydroxyl, amino or methoxy group,

R₄ is a hydrogen atom, a hydroxyl group, a methoxy group or C₁-C₄alkyl group,

with the proviso that:

when R₃ is an amino group, it is in position 4,

when R₃ is in position 4, R₄ is in position 7,

when R₃ is in position 5, R₄ is in position 6.

Among the benzimidazole derivatives of the above formula (I) the following can in particular be mentioned: 4-hydroxy benzimidazole, 4-amino benzimidazole, 4-hydroxy-7-methyl benzimidazole, 4-hydroxy-2-methyl benzimidazole, 1-butyl-4-hydroxy benzimidazole, 4-amino-2-methyl benzimidazole, 5,6-dihydroxy benz-imidazole, 5-hydroxy-6-methoxy benzimidazole, 4,7-dihydroxy benzimidazole, 4,7-dihydroxy-1-methyl benzimidazole, 4,7-dimethoxy benzimidazole, 5,6-dihydroxy-1-methyl benzimidazole, 5,6-dihydroxy-2-methyl benzimidazole, 5,6-dimethoxy benzimidazole and their addition salts with an acid.

Among the benzomorpholine derivatives applicable as heterocyclic coupling agents in the ready-to-use dye composition according to the invention, the compounds of the following formula (II) and their addition salts with an acid can in particular be mentioned:

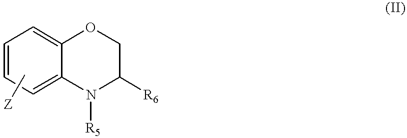

in which

R₅ and R₆, which are identical or differ, each represents a hydrogen atom or C₁-C₄-alkyl, and

Z represents a hydroxyl group or an amino group.

Among the benzomorpholine derivatives of the above formula (II) the following can in particular be mentioned: 6-hydroxy 1,4-benzomorpholine, N-methyl 6-hydroxy 1,4-benzomorpholine, 6-amino 1,4-benzomorpholine and their acid addition salts.

Among the derivatives of sesamol applicable as heterocyclic coupling agents in the ready-to-use dye composition according to the invention, the compounds of the following formula (III) and their addition salts with an acid can in particular be mentioned:

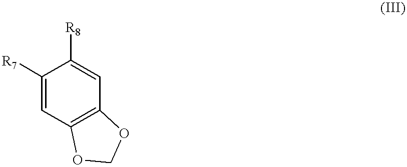

in which

R₇ represents a hydroxyl group, an amino group, a C₁-C₄-alkylamino group, a C₁-C₄monohydroxyalkylamino group or a C₂-C₄polyhydroxyalkylamino group,

R₈ represents a hydrogen atom, a halogen atom or a Cl-₄alkoxy group.

Among the derivatives of sesamol of the above formula (III), the following can in particular be mentioned: 2-bromo 4,5-methylenedioxy phenol, 2-methoxy 4,5-methylenedioxy aniline, 2-(β-hydroxyethyl)amino 4,5-methylenedioxy benzene and their acid addition salts.

Among the pyrazolo-azole derivatives applicable as heterocyclic coupling agents in the ready-to-use dye composition according to the invention, the compounds can in particular be mentioned which are described in the following Patents and Patent Applications: FR 2 075 583, EP-A-119 860, EP-A-285 274, EP-A-244 160, EP-A-578 248, GB 1 458 377, U.S. Pat. No. 3,277,554, U.S. Pat. No. 3,419,391, U.S. Pat. No. 3,061,432, U.S. Pat. No. 4,500,630, U.S. Pat. No. 3,725,067, U.S. Pat. No. 3 926 631, U.S. Pat. No. 5,457,210, JP 84/99437, JP 83/42045, JP 84/162548, JP 84/171956, JP 85/33552, JP 85/43659, JP 85/172982, JP 85/190779 as well in the following publications: Chem. Per. 32, 797, (1899), Chem. Ber. 89, 2550, (1956), J. Chem. Soc. Perkin trans I, 2047, (1977), J. Prakt. Chem., 320, 533, (1978), the subject matter of which constitute an integrated part of the present application.

As the pyrazolo-azole derivatives, the following can in particular be mentioned:

2-methyl pyrazolo[1,5-b]-1,2,4-triazole,

2-ethyl pyrazolo[1,5-b]-1,2,4-triazole,

2-isopropyl pyrazolo[1,5-b]-1,2,4-triazole,

2-phenyl pyrazolo[1,5-b]-1,2,4-triazole,

2,6-dimethyl pyrazolo[1,5-b]-1,2,4-triazole,

7-chloro-2,6-dimethylpyrazolo[1,5-b]-1,2,4-triazole,

3,6-dimethyl-pyrazolo[3,2-c]-1,2,4-triazole,

6-phenyl-3-methylthio-pyrazolo[3,2-c]-1,2,4-triazole,

6-amino-pyrazolo[1,5-a]benzimidazole,

and their addition salts with an acid.

Among the pyrrolo-azole derivatives applicable as heterocyclic coupling agents in the ready-to-use dye composition according to the invention, the compounds can in particular be mentioned which are described in the following Patents and Patent Applications: U.S. Pat. No. 5,256,526, EP-A-557 851, EP-A-578 248, EP-A-518 238, EP-A-456 226, EP-A-488 909, EP-A-488 248 and in the following publications:

D.R. Liljegren Ber. 1964, 3436;

E.J. Browne, J.C.S., 1962, 5149;

P. Magnus, J.A.C.S., 1990, 112, 2465;

P. Magnus, J.A.C.S., 1987, 109, 2711;

Angew. Chem. 1960, 72, 956; and

Rec. Trav. Chim. 1961, 80, 1075, the subject matter of which constitute an integrated part of the present application.

As the pyrazolo-azole derivatives, the following can in particular be mentioned:

5-cyano-4-ethoxycarbonyl-8-methyl pyrrolo [1,2-b]-1,2,4-triazole,

5-cyano-8-methyl-4-phenyl pyrrolo [1,2-b]-1,2,4-triazole,

7-amido-6-ethoxycarbonyl pyrrolo [1,2-a]-benzimidazole, and their addition salts with an acid.

Among the imidazolo-azole derivatives applicable as heterocyclic coupling agents in the ready-to-use dye composition according to the invention, the compounds can in particular be mentioned which are described in the following Patents and Patent Applications: U.S. Pat. No. 5,441,863, JP 62-279 337, JP 06-236 011 and JP 07-092 632, the subject matter of which constitute an integrated part of the present application.

As the imidazolo-azole derivatives, the following can in particular be mentioned:

7,8-dicyano-imidazolo-[3,2-a]-imidazole,

7,8-dicyano-4-methyl-imidazolo-[3,2-a]-imidazole, and their addition salts with an acid.

Among the pyrazolo-pyrimidine derivatives applicable as heterocyclic coupling agents in the dye composition according to the invention, the compounds can in particular be mentioned which are described in the following Patent Application: EP-A-304-001, the subject matter of which constitute an integrated part of the present application.

As the pyrazolo-pyrimidine derivatives, the following can in particular be mentioned:

pyrazolo-[1,5-a]-pyrimidine-7-one,

2,5-dimethyl pyrazolo [1,5-a] pyrimidine-7-one,

2-methyl-6-ethoxycarbonyl pyrazolo [1,5-a] pyrimidine-7-one,

2-methyl-5-methoxymethyl pyrazolo [1,5-a] pyrimidine-7-one,

2-tert-butyl-5-trifluoromethyl pyrazolo [1,5-a] pyrimidine-7-one,

2,7-dimethyl pyrazolo [1,5-a] pyrimidine-5-one, and their addition salts with an acid.

Among the pyrazoline-3,5-diones derivatives applicable as heterocyclic coupling agents in the ready-to-use dye composition according to the invention, the compounds can in particular be mentioned which are described in the following Patents and Patent Applications: JP 07-036159, JP 07-084348 and US 4.128.425, and in the following publications:

L. WYZGOWSKA, Acta. Pol. Pharm. 1982, 39 (1-3), 83.

E. HANNIG, Pharmazie, 1980, 35 (4), 231

M. H. ELNAGDI, Bull. Chem. Soc. Jap., 46(6), 1830, 1973

G. CARDILLO, Gazz. Chim. Ital. 1966, 96, (8-9), 973,

the subject matter of which constitute an integrated part of the present application.

As the derivatives of pyrazolin-3,5-diones, the following can in particular be mentioned:

1,2-diphenyl pyrazoline-3,5-dione,

1,2-diethyl pyrazoline-3,5-dione,

and their addition salts with an acid.

Among the pyrrolo-[3,2-d]-oxazole derivatives applicable as heterocyclic coupling agents in the ready-to-use dye composition according to the invention, the compounds can in particular be mentioned which are described in the Patent Application JP 07-325,375, the subject matter of which constitute an integrated part of the present application.

Among the pyrazolo-[3,4-d]-thiazole derivatives applicable as heterocyclic coupling agents in the ready-to-use dye composition according to the invention, the compounds can in particular be mentioned which are described in the Patent Application JP 07-244,361 and in J. Heterocycl. Chem. 16, 13, (1979).

Among the thiazolo-azole S-oxide derivatives and thiazolo-azole S,S-dioxide derivatives applicable as heterocyclic coupling agents in the ready-to-use dye composition according to the invention, the compounds can in particular be mentioned which are described in the following documents:

JP 07 09 84 89;

Khim. Geterotsilk. Soedin, 1967, p. 93;

J. Prakt. Chem., 318, 1976, p. 12;

Indian J. Heterocycl. Chem. 1995, 5(2), p. 135;

Acta. Pol. Pharm. 1995, 52(5), 415;

Heterocycl. Commun. 1995, 1(4), 297;

Arch. Pharm. (Weinheim, Ger.), 1994, 327(12), 825.

These coupling agents constitute preferably between approximately 0.0001 and approximately 10% by weight of the ready-to-use dye composition, especially between approximately 0.005 and approximately 5% by weight.

Direct Dyes

The dyeing composition according the invention may also contain direct dyes.

The cationic direct dye(s) applicable in the dye composition according to the invention is/are preferably selected among cationic amino-anthraquinone dyes, cationic mono or di-azo dyes and cationic naphtoquinone dyes.

Examples of the above are especially[8-[(p-aminophenyl)azo]-7-hydroxy-2-naphtyl]trimethylammonium chloride (also called Basic Brown 16 or Arianor Mahogany 306002 in Color Index), 3-[(4-amino-6-bromo-5,8-dihydro-1-hydroxy-8-imino-5-oxo-2-naphtalenyl)amino]-N,N,N-trimethyl-benzeneaminium chloride (also called Basic Blue 99 or Arianor Steel Blue 306004 in Color Index), 7-hydroxy-8-[(2-methoxyphenyl)azo]-N,N,N-trimethyl-2-naphtaleneaminium chloride (also called Basic Red 76 or Arianor Madder Red in Color Index), [8-[(4-amino-2-nitrophenyl)azo]-7-hydroxy-2-naphtyl]trimethylammonium chloride (also called Basic Brown 17 or Arianor Sienna Brown 306001 in Color Index) and 3-[(4,5-dihydro-3-methyl-5-oxo-1-phenyl-1H-pyrazol-4-yl)azo]-N,N,N-trimethylbenzenaminium chloride (also called Basic Yellow 57 or Arianor Straw Yellow 306005 in Color Index).

The cationic direct dye(s) can furthermore be selected among:

a) Compounds of the formula (V):

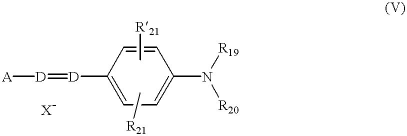

in which

D is a nitrogen atom or a group —CH,

R₁₉ and R₂₀, which are identical or differ, each is a hydrogen atom, a C₁-C₄alkyl group, which can be substituted with one of the groups —CN, —OH or —NH₂ or together with a carbon atom in the benzene ring form an optionally oxygen-containing or nitrogen-containing heterocyclic group, which can be substituted with one or more C₁-C₄alkyl groups; or a 4′-aminophenyl group,

R₂₁ and R′₂₁, which are identical or differ, each is a hydrogen atom or a halogen atom selected from chlorine, bromine, iodine and fluorine, cyano, C₁-C₄-alkoxy or acetyloxy,

X⁻ is an anion, preferably selected from chloride, methylsulphate and acetate,

A is a group selected from the following structures A1-A19:

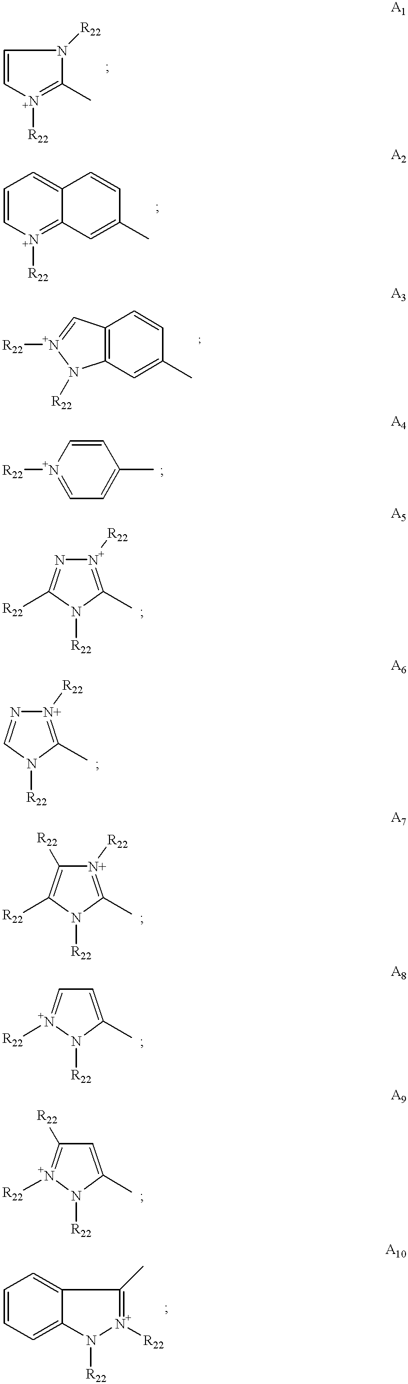

wherein R₂₂ is a C₁-C₄alkyl group, which can be substituted with a hydroxyl group, and R₂₃ represents a C₁-C₄alkoxy group;

b) compositions of the formula (VI):

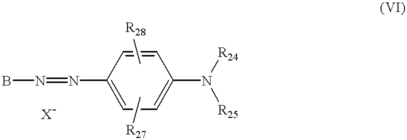

in which

R₂₄ is a hydrogen atom or a C₁-C₄alkyl group,

R₂₅ is a hydrogen atom, an alkyl group, which can be substituted with a group —CN or with an amino group, or 4′-aminophenyl, or R₂₅ represents together with R₂₄ an optionally oxygen and/or nitrogen-containing heterocyclic group, which can be substituted with a C₁-C₄alkyl group, R₂₆ and R₂₇, which are identical or differ, are a hydrogen atom, a halogen atom such as bromine, chlorine, iodine or fluorine, C₁-C₄alkyl, C₁-C₄alkoxy or the group —CN,

X⁻ is an anion, preferably selected from chloride, methylsulphate and acetate,

B is a group selected from the following structures B1-B6:

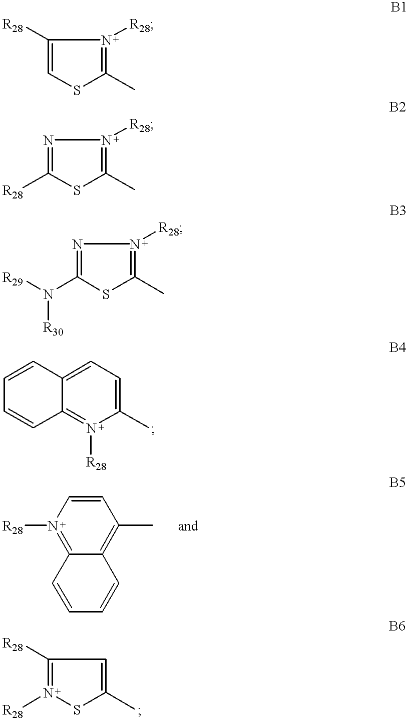

in which R₂₈ is a C₁-C₄alkyl group, and R₂₉ and R₃₀, which are identical or differ, each is a hydrogen atom or a C₁-C₄alkyl group;

c) compounds of the following formulae (VII) and (VII′):

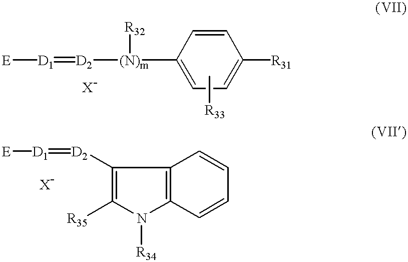

in which

R₃₁ is a hydrogen atom, a C₁-C₄alkoxy group, a halogen atom such as bromine, chlorine, iodine or fluorine, or an amino group,

R₃₂ is a hydrogen atom or a C₁-C₄alkyl group, or R₃₂ together with a carbon atom in the benzene ring forms a heterocyclic group, which optionally includes an oxygen atom and/or is substituted with one or more C₁-C₄alkyl groups,

R₃₃ is a hydrogen atom or a halogen atom such as bromine, chlorine, iodine or fluorine,

R₃₄ and R₃₅, which are identical or differ, each is a hydrogen atom or a C₁-C₄alkyl group,

D₁ and D₂, which are identical or differ, are a nitrogen atom or a group —CH,

m=0 or 1,

whereby it should be understood that when R₃₁ represents a non-substituted amino group, D₁ and D₂ are simultaneously a group —CH, and m=0,

X⁻ is an anion, preferably selected from chloride, methylsulphate and acetate,

E is a group selected from the following structures E1-E8:

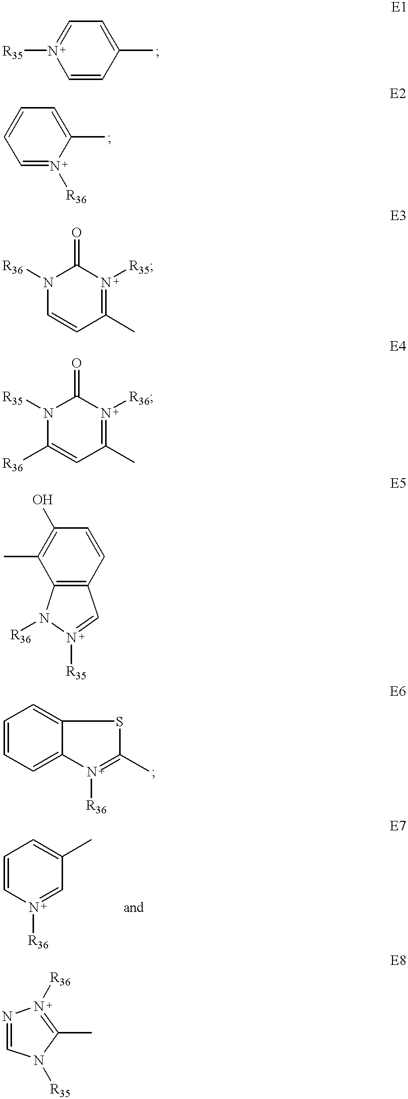

in which R₃₆ is a C₁-C₄alkyl group;

when m=0 and D₁ is a nitrogen atom, E can also represent a group with the following structure E9:

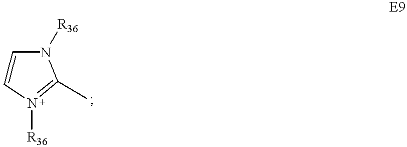

in which R₃₆ is a C₁-C₄alkyl group.

The cationic direct dyes of the formulae (V), (VI), (VII) and (VII′), which are applicable in the ready-to-use dye compositions according to the invention, are compositions known per se, which are described for instance in the Patent Applications WO 95/01772, WO 95/15144 and EP-A-0 714 954.

Among the cationic direct dyes of the formula (V), which are applicable in the ready-to-use dye compositions according to the invention, the compounds of the following structures (VI) to (V52) can in particular be mentioned:

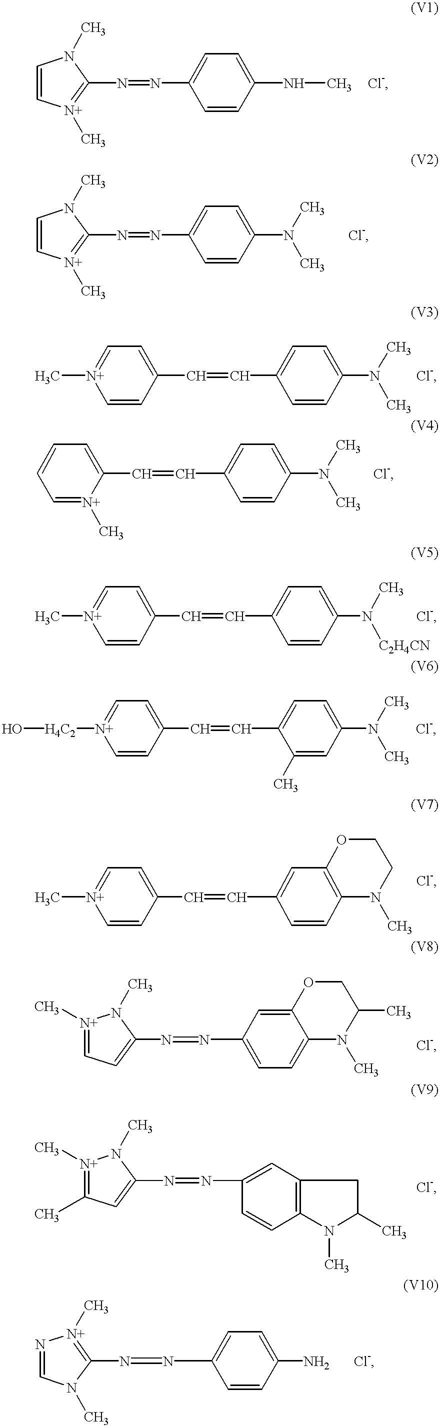

Among the above compounds with the structures (V1) to (V52), the compounds with the structures (V1), (V2), (V4), (Vl4) and (V31) are particularly preferred.

Among the cationic direct dyes of the formula (VI), which are applicable in the ready-to-use dye compositions according to the invention, the compounds with the following structures (VII) to (VI12) can in particular be mentioned:

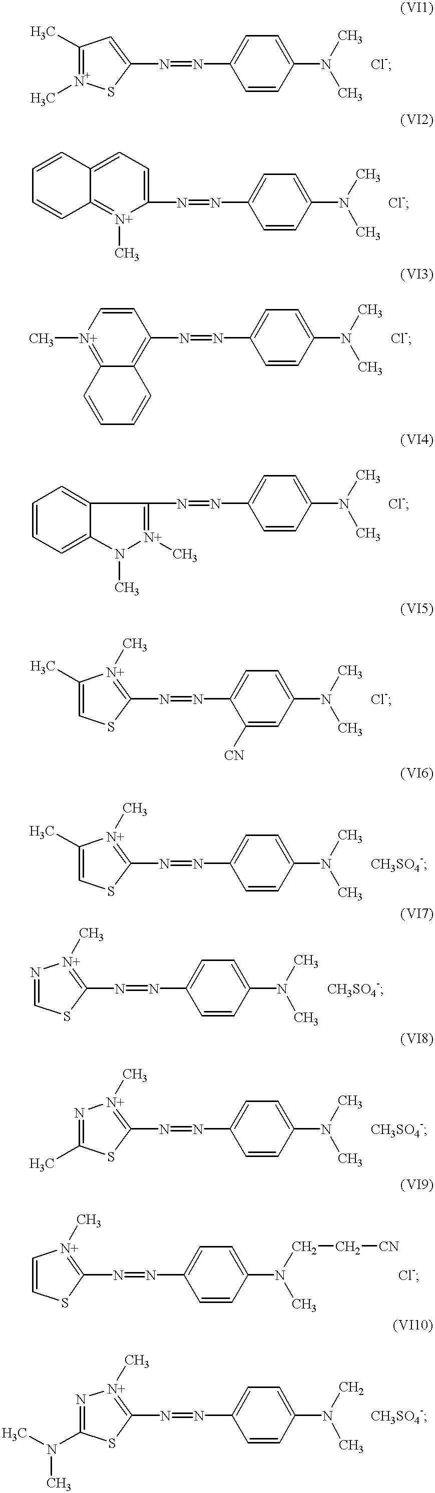

Among the cationic direct dyes of the formula (VII), which are applicable in the ready-to-use dye compositions according to s the invention, the compounds with the following structures (VI11) to (VI18) can in particular be mentioned:

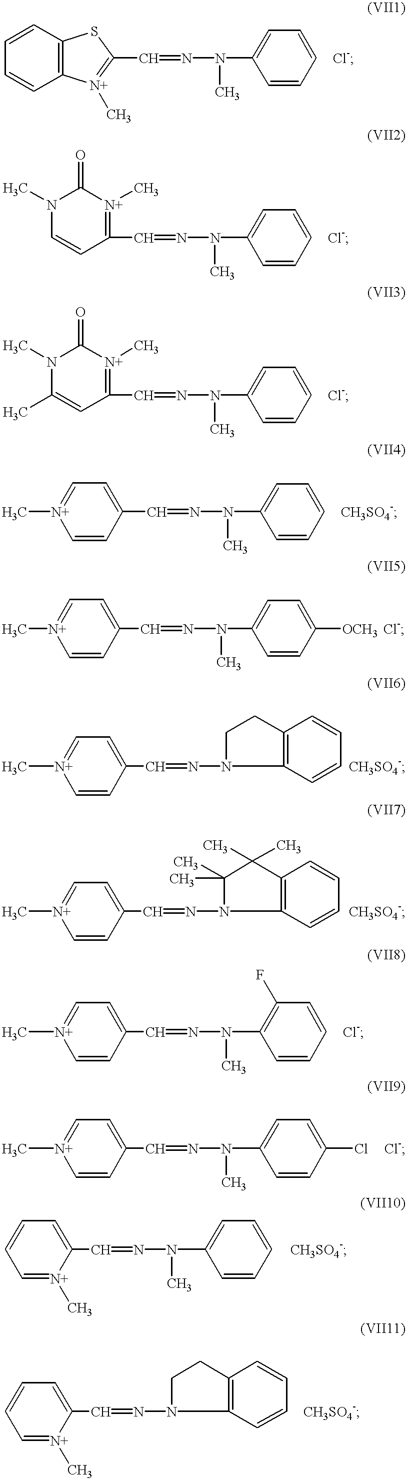

Among the above particular compositions with the structures (VIII) to (VII18), the compounds with the structures (VII4), (VII5) and (VII13) are particularly preferred.

Among the cationic direct dyes of the formula (VII′), which are applicable in the ready-to-use dye compositions according to the invention, the compounds with the following structures (VII′1) to (VII′3) can in particular be mentioned:

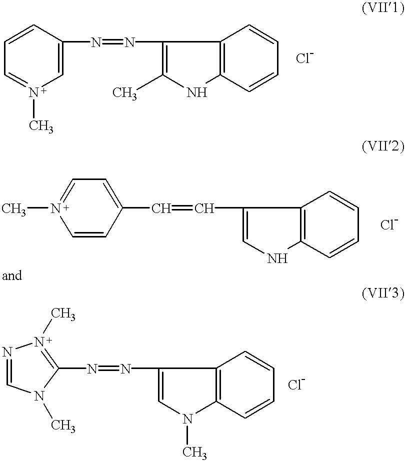

The cationic direct dye or dyes used according to the invention represent preferably between approximately 0.001 and approximately 10% by weight of the total weight of the ready-to-use dye composition, especially between approximately 0.05 and approximately 5% by weight.

In general, the acid addition salts suitable within the scope of the dye compositions according to the invention (oxidation bases and coupling agents) are especially selected from hydrochlorides, hydrobromides, sulphates, tartrates, lactates and acetates.

Compositions

The proper environment for the colouring (or support) of the colouring composition corresponding to the invention is generally composed of water or a mixture of water and at least an organic solvent to dissolve the components that are not sufficiently soluble in water. As an example of an organic solvent one can mention C₁-C₄ alcanols, such as ethanol and isopropanol as well as aromatic alcohols like benzyl alcohol, and analogue products and their mixtures.

The solvents can be present in quantities preferably between 1 and 40% of the total weight of the colouring composition and rather between 5 and 30% of the weight. The pH of the composition corresponding to the invention is chosen in a way that ensures a sufficient enzymatic activity of the laccase. The pH generally lies between 3 and 10, preferably between 5 and 9, especially between 6 and 8.

The colouring composition corresponding to the invention can is also contain different additives typically used in hair colouring compositions, such as anionic, cationic, non-ionic, amphoteric or zwitterionic tensio-active agents or their mixtures, polymers, thickening agents, antioxidants, penetration agents, sequestrant agents, perfumes, buffers, dispersion agents, filmogene agents, filtration agents, vitamins, preservation agents and opacity agents.

Of course a person skilled in the art will be careful to choose the possible complementary components in a way that the advantageous properties of the colouring composition corresponding to the invention are not, or not substantially, changed by the foreseen adjunctions.

The colouring composition corresponding to the invention can have different forms, such as liquids, creams, gels, maybe pressurized, or any other form that is appropriate for colouring keratinous fibers, and especially human hair. In this case the oxidation colour or colours and the enzymes are present in the same composition, and consequently the mentioned composition must be free of gaseous oxygene in order to avoid any premature oxidation of the oxidation colour or colours.

The invention has also as an objective a colouring procedure of keratinous fibers and especially human keratinous fibers such as hair, implementing the colouring composition such as defined above.

According to this procedure, at least one colouring composition such as defined earlier is applied to the fibers, for a time that is sufficient to develop the desired coloration, whereafter the fibers are rinsed, if necessary washed with a shampoo, rinsed again, and dried.

The time necessary for developing the coloration of the keratinous fibers generally lies between 10 and 60 minutes, preferably between 15 and 50 minutes and more preferably between 20 and 40 minutes.

According to a special realisation form of the invention the procedure includes a preliminary stage consisting in stocking in separate form, on one side, a composition (A) comprising, in an environment appropriate for colouring, at least one oxidation colour, at least one direct cationic colour and, on the other side, a composition (B) including, in an environment appropriate for colouring, at least one enzyme, than proceeding with the mixing of these at the moment of use before the mixture is applied to the keratinous fibers.

Another object of the invention is a device with more compartments or a colouring kit or any other container system with more compartments, of which a first compartment contains the composition (A) as defined above and a second compartment contains the composition (B) as defined above. These devices can be equipped with means permitting to apply the desired mixture to the hair, as the devices described in the FR-2 586 913.

By adding small amounts (i.e., 0.001-1%, preferably 0.01-0.5%) of a hydrogen peroxide source along with at least one oxidoreductase and one or more mediators, the efficiency of dyeing increases substantially, while no significant damage to the hair is observed.

Special Compositions

In the case of an enzyme acting on oxygen (02) as the acceptor, said oxygen may be molecular oxygen supplied by the air. In a preferred embodiment, part of the oxygen is provided by a foam produced from a hair setting/hair dyeing composition comprising a foaming agent.

Suitable enzymatic foam compositions for hair dyeing which may be used according to the invention include hair-dyeing compositions that comprise foaming agent selected from soaps and anionic, cationic, non-ionic, amphoteric, sugar surfactants and/or zwitterionic surfactants and mixtures thereof. The foaming agent(s) may be present at levels of from 0.1% to 15%, preferably from 0.2 to 13%, more preferably from 0.25 to 10%, e.g., from 0.5 to 8% by weight of the final composition. Examples of anionic surfactants suitable for use as the foaming agent are soaps, e.g., in the form of alkali or ethanolamine, isopropanol 2-methyl-2-amino-1,3-propanediol salts of fatty acids such as laurate, myristate, palmitate, stearate, isostearate, behenate, oleate, linoleate, etc.; fatty alcohol ether sulfates such as sodium lauryl ether sulfate; fatty alcohol sulfates such as sodium lauryl sulfate (SLS and SDS); sulfo succinates, e.g. dioctyl sodium sulfo succinate; α-olefin sulfonates; alkyl amide ether sulfates; fatty acid condensation products; alkyl ether phosphates and monoglyceride sulfates. Examples of non-ionic surfactants suitable for use as the foaming agent are especially the nonionic fatty acids and fatty amines that often are used as foam stabilizers, thickeners and boosters, e.g. fatty acid alkanol amides and dialkanol amides and fatty acid alkanol amide polyglycol ethers and fatty amine oxides. Examples of amphoteric surfactants suitable for use in combination with anionic surfactants as the foaming agent are alkyl betaines, alkyl imidazolinium betaines, alkyl sulfo betaines, amidoalkyl betaines, N-alkyl-β-amino propionates, etc.

Examples of foaming agents in the form of sugar surfactants include (a) alkyl- and/or alkenyloligoglycosides and/or (b) fatty acid-N-alkylpolyhydroxyalkylamides. The alkyl- and/or alkenyloligoglycoside (a) has the formula:

R1—O—[G]p (I),

in which R1=4-22C alkyl and/or alkenyl group, G=a sugar residue with 5 or 6C and p=1-10. The fatty acid-N-alkylpolyhydroxyalkylamide (b) has the formula:

R2CO—N(R3)—[Z] (II),

in which R2CO=a 6-22C aliphatic acyl residue, R3=H, alkyl or hydroxyalkyl with 1-4C and [Z]=a linear or branched polyhydroxyalkyl residue with 3-12C and 3-10 OH groups;

a) alkyl and alkenyl oligoglycosides of formula R1—O[G]p (I) and b) alkali and/or alkali metal salts of 12-22C secondary 2,3-alkyl sulphates (II). R1=4-22C alkyl and/or alkenyl; G=5-6C sugar residue; p=1-10. The wt. ratio (I):(II) is pref. 1:99-99:1; and

(A) fatty acid-N-alkyl polyhydroxyalkyl amides; and

(B) sugar surfactants of: (B1) saccharose esters, (B2) sorbitan esters and/or (B3) polysorbates.

A sugar surfactant may also comprise 10-40% (wt.) alkyl and/or alkenyl-oligoglucoside of the formula

R1—O—[G]p (II),

10-40% alkyl- and/or alkenyl-oligoglucoside of the formula R2—O—(G)p (III),

and 80-20% alkyl ether sulphate of the formula

R3—(OCH2CH2)nO—SO3M (IV)

in which R1=8-11C alk(en)yl; (G)=a glucose gp.; p=1-10; (1-3) R2=12-22C alk(en)yl; R3=6-22C alk(en)yl; M=an alkali(ne earth), ammonium or alkanolammonium ion; (pref. Na, Mg) n=1-20 2-7. Pref. R2, R3=12-14C alkyl; and polyglycerine fatty acid ester polyoxyalkylene ether RR1R2R3N+—CH(Y)—CH2—O—CH2—C(CH3)2—C(OH) (H)—C(═O)—NH—CH2—CH2—OH X—(I) where R, R1, R2=1-24C alkyl or 8-24C alkenyl; R3=1-18C alkylene; X=monovalent (in)organic anion; and Y=OH or H; and 1-5 wt. % of fatty alcohol polyglycol ether, 1-5% of Guerbet alcohol, 1-5% of polyol partial ester, (B) 1-5% of anionic polymer, (C) 15-30% of fatty alcohol polyglycol ether sulphate, (D) 15-30% of alkyloligoglycoside; and sulphated prods. of fatty acid-N-alkylpolyhydroxyalkyl amides of formula R1CO—N(R2)—Z (I), R1CO=6-22C aliphatic acyl; R2=H, 1-4C alkyl or 1-4C hydroxyalkyl; Z=3-12C polyhydroxyalkyl contg. 3-10 hydroxy gps; and sugar surfactant solubilisers selected from alkyl oligoglycosides of formula (I) and carboxylic acid N-polyhydroxyalkylamides of formula (II). R1—O(G)p (I) R2CO—NR3—Z (II) R1=opt. hydroxylated 1-8C alkyl; G=5C or 6C sugar residue; p=1-10; R2CO=1-8C aliphatic acyl; R3=H, 1-8C alkyl or 1-8C hydroxyalkyl; Z=3-12C polyhydroxyalkyl contg. 3-10 OH gps.

Examples of preferred foaming agents are SDS (sodium dodecyl sulfate), sodium dodecyl ether sulfate and soaps.

It may also be desired to add other additives that function as stabilizers, boosters and thickeners, for example one or more compounds selected from fatty acid alkanol amides, dialkanol amides or fatty alkanol amides, polyglycol ethers such as ethoxylated lauric acid monoethanol amide, or fatty amine oxides such as alkyl dimethyl amine oxide. In connection with an anionic surfactants such as SDS, it will often be preferred to use an amphoteric surfactant such as betaine phosphate.

### MATERIALS AND METHODS

Materials:

Enzyme

Laccase solution: Myceliophthora thermophila laccase (MtL) 5 described in WO 95/33836 (Novo Nordisk), stock solution, 0.05 mg ep/ml (ep=active enzyme protein).

Dye Mixtures

Dye mixture 1:

PPD 0.3%

Dye mixture 2:

PPD 0.3%

5-amino-o-cresol 0.3%

Dye mixture 3:

PTD 0.3%

OAP 0.3%

5-amino-o-cresol 0.3% and

MAP 0.3%

Dye mixture 4:

TAP 0.07146%

PTD 0.4406%

Methylyellow 0.4928%

2-methylresorcin 0.3971%

2,7 dihydroxynaphtalin 0.16%

4-chlorresorcin 0.1012%

2-amino-3-hydroxypyridin 0.4404% and

Rodol 9R base 0.1%

All dye mixtures is combined the laccase so that the final concentration of laccase in the dye mixture is 0.05mg ep/ml

Buffer

0.1 M K-phosphat pH 7.0

Methods:

Determination of Laccase Activity (LAMu)

The LAMU method is used for determining the activity of Myceliophthora thermophila laccase. 1 laccase unit (LAMU) is the amount of enzyme which catalyses the conversion of 1.0 micro mole syringaldazine per minute under the following analytical conditions. Further details on how to determine LAMU can be found in WO 98/40471 (see pages 18 to 20) (Novo Nordisk).

Assessment of the Hair Colour

The quantitative colour of the hair tresses is determined on a Minolta CR200 Chroma Meter by the use the parameters L* (“0”=black and “100”=white), a* (“−”=green and “+”=red) and b* (“−” blue and “+” yellow).

ΔL*, Δa* and Δb* are the delta values of L*, a* and b* respectively compared to L*, a* and b* of untreated hair (e.g. ΔL*=L*ₛₐₘₚₗₑ−L*_{untreated hair}).

ΔE* is calculated as ΔE*=√(ΔL*²+Δa*²+Δb*²) and is an expression for the total quantitative colour change.

### EXAMPLES

### Example 1

2 samples of hair (obtained from Bertello Aps, each sample contained about 1 g hair) were prepared by fusing the root ends with adhesive. Half of the samples were subjected to a wetting treatment by soaking in water for about 15 minutes, while the other half were left untreated. Thereafter, the samples were subjected to a dyeing process by applying the different dye mixtures. This is done by placing each wisp of hair in a beaker adding the dye solution mixed with the laccase, and incubated at 30° C. for 30 minutes. During the incubation, the beakers are shaken at 150 r.p.m. The hair is rinsed for 3 minutes under running water, followed by air-drying at room temperature. Assessment of colour was carried out.

Results are shown in table 1 below.

TABLE 1
Delta E for Bertello dry and wet hair

| | Dry | Wet |
|---|---|---|
| dyemix 4 | 48,32 | 45,78 |

### Example 2

The procedure of example 1 were carried out with 8 samples of hair (obtained from De Meo Brothers, Inc., each sample contained about 1.00 g hair) prepared by fusing the root ends with adhesive.

Results are shown in table 2 and FIG. 1.

TABLE 2
Delta E for DeMeo dry and wet hair

| | dry | Wet |
|---|---|---|
| dyemix 1 | 47,21 | 46,16 |
| dyemix 2 | 39,18 | 37,12 |
| dyemix 3 | 39,21 | 38,61 |
| dyemix 4 | 36,06 | 35,66 |

In summary, dyeing of hair can be improved by dyeing the hair when it's originately dry instead of dyeing hair that is previously wetted.

1. A method for dyeing keratinous fibers comprising contacting the fibers in a dry state with a dyeing composition comprising at least one oxidoreductase and at least one dye precursor for a sufficient period of time and under conditions sufficient to permit dyeing of the fibers.

2. The method of claim 1, wherein the oxidoreductase is of microbial origin.

3. The method of claim 2, wherein the oxidoreductase is of bacterial, filamentous fungal or yeast origin.

4. The method of claim 1, wherein the at least one oxidoreductase is an oxidase or a peroxidase, or a mixture thereof.

5. The method of claim 1, wherein the oxidoreductase is a laccase.

6. The method of claim 5, wherein the oxidoreductase is a laccase derived from Myceliophthora sp.

7. The method of claim 6, wherein the oxidoreductase is a laccase derived from M. thermophila.

8. The method of claim 1, wherein the dyeing composition further comprises a mediator.

9. A method of claim 8, wherein the mediator is selected from the group consisting of m-diamines, m-aminophenols and polyphenols, and mixtures thereof.

10. The method of claim 8, wherein the mediator is selected from the group consisting of 2,2′-azino-bis(3-ethylbenzothiazoline-6-sulfonate (ABTS), 6-hydroxy-2-naphtoic acid, 7-methoxy-2-naphtol, 7-amino-2-naphthalene sulfonic acid, 5-amino-2-naphthalene sulfonic acid, 1,5-diaminonaphthalene, 7-hydroxy-1,2-naphthimidazole, 10-methylphenothiazine, 10-phenothiazine-propionic acid (PPT), N-hydroxysuccinimide-10-phenothiazine-propionate, benzidine, 3,3′-dimethylbenzidine, 3,3′-dimethoxybenzidine, 3,3′,5,5′-tetramethylbenzidine, 4′-hydroxy-4-biphenylcarboxylic acid, 4-amino-4′-methoxystilbene, 4,4′-diaminostilbene-2,2′-disulfonic acid, 4,4′-diaminodiphenylamine, 2,7-diaminofluorene, 4,4′-dihydroxy-biphenylene, triphenylamine, 10-ethyl-4-phenothiazinecarboxylic acid, lo-ethylphenothiazine, 10-propylphenothiazine, 10-isopropylphenothiazine, methyl-10-phenothiazinepropionate, 10-phenylphenothiazine, 10-allylphenothiazine, 10-phenoxazinepropionic acid (POP), 10-(3-(4-methyl-1-piperazinyl)propyl)phenothiazine, 10-(2-pyrrolidinoethyl)phenothiazine, 10-methylphenoxazine, iminostilbene, 2-(p-aminophenyl)-6-methylbenzothiazole-7-sulfonic acid, N-benzylidene-4-biphenylamine, 5-amino-2-naphthalenesulfonic acid, 7-methoxy-2-naphtol, 4,4′-dihydroxybenzophenone, N-(4-(dimethylamino)benzylidene)-p-anisidine, 3-methyl-2-benzothiazolinone(4-(dimethylamino)benzylidene)hydrazone, 2-acethyl-10-methylphenothiazine, 10-(2-hydroxyethyl)phenothiazine, 10-(2-hydroxyethyl)phenoxazine, 10-(3-hydroxypropyl)phenothiazine, 4,4′-dimethoxy-N-methyl-diphenylamine, vanillin azine, 4-hydroxybenzoic acid, L-tyrosine, syringate acids, ferulic acid, sinapic acid, chlorogenic acid, caffeic acid and esters thereof, acetosyringone, syringaldehyde, methylsyringate, syringic acid, ethylsyringate, propylsyringate, butylsyringate, hexylsyringate, octylsyringate and ethyl 3-(4-hydroxy-3,5-dimethoxyphenyl)acrylate, or a combination thereof.

11. The method of claim 1, wherein the precursor is selected from the group consisting of diamines, aminophenols, pyridines, pyrimidines, pyrazoles and pyrazole pyrimidines derivatives.

12. The method of claim 1, which is carried out at a pH in the range from 3 to 10.

13. The method of claim 12, which is carried out at a pH in the range from 5 to 9.

14. The method of claim 13, which is carried out at a pH in the range from 6 to 8.

15. The method of claim 1, which is carried out for a period of time between 10 and 60 minutes.

16. The method of claim 15, which is carried out for a period of time between 15 and 50 minutes.

17. The method of claim 16, which is carried out for a period of time between 20 and 40 minutes.
